(19) United States
(12) Patent Application Publication
(10) Pub. No.: US 2025/0040836 A1
(43) Pub. Date: Feb. 6, 2025

(54) CONTINUOUS EXTRACTION AND SENSING OF INTERSTITIAL FLUID

(71) Applicant: University Of Cincinnati, Cincinnati, OH (US)

(72) Inventors: Jason Charles Heikenfeld, Cincinnati, OH (US); Mark Friedel, Cincinnati, OH (US)

(21) Appl. No.: 17/421,794

(22) PCT Filed: Nov. 13, 2019
(86) PCT No.: PCT/US2019/061123

§ 371 Date: Jul. 9, 2021

Related U.S. Application Data
(60) Provisional application No. 62/791,401, filed on Jan. 11, 2019.

(51) Int. Cl. A61B 5/145; A61B 5/1455; A61B 5/1477; A61B 5/15

(52) U.S. Cl. CPC A61B 5/14514; A61B 5/1455; A61B 5/1477; A61B 5/150099; A61B 2560/0462

(57) ABSTRACT

Described are sensing devices and methods that continuously sense an analyte included in an interstitial fluid. The device includes at least one ex-vivo sensor specific to the a least one analyte in interstitial fluid. The device further includes at least one sample collection component in the dermis that defines at least in part an advective pathway to transport interstitial fluid to the at least one sensor. The advective pathway is air-tight, and at least one integrated pump applies negative pressure to cause advective transport of interstitial fluid from the dermis, to the sensor, and onto the pump.

### CROSS-REFERENCE TO RELATED APPLICATIONS

This application claims priority to, and the benefit of the filing date of, U.S. Provisional Application No. 62/791,401 filed Jan. 11, 2019, the disclosure of which incorporated by reference herein in its entirety.

### BACKGROUND OF THE INVENTION

Interstitial fluid contains many of the same analytes as blood and often at comparable concentrations. As a result, interstitial fluid (ISF) presents an alternate biofluid to blood for detection of analytes such as glucose for diabetes monitoring. Commonly employed practices for continuous monitoring of glucose in interstitial fluid include in-dwelling sensors, where a needle is utilized to insert the sensor into the dermis of the skin, and micro-needles where the sensor is placed ex-vivo and the analyte is coupled from interstitial fluid to the sensor by diffusion to the sensor. In both products and in research, the biosensing of analytes in interstitial fluid monitoring has been dominated by devices that rely on diffusion of analytes to a sensor without significant impact by advective transport of analytes to a sensor. Interstitial fluid monitoring has been dominated by enzymatic sensing of metabolites, which works well even with ex-vivo diffusion-based sensing because the enzymatic sensors consume (convert the analyte), which makes them responsive to diffusive flux to the sensor, a diffusive flux which changes immediately with changes in concentration of the analyte in ISF. As a result, for enzymatic sensors and metabolites, there previously has been no purpose or advantage in using advective flow to transport the analyte to the sensor.

Utilizing continuous advective flow (continuous ISF extraction) is generally a more complex approach than a simple diffusion-only based device scheme. In practice, such demonstrations have been limited to devices which perform single sample extractions, using unsustainable techniques such as positive pressure which collapses the dermis. Furthermore, if even a continuous ISF extraction and sensing device were built and tested using existing methods for ISF extraction, it would be difficult if not prohibitive to make a practically useful device due to the many drawbacks/limitations of current approaches for ISF extraction. This is unfortunate, because in some cases ISF extraction will be required, for example to reduce lag time for slowly-diffusing components with affinity-based biosensors, to implement sample pre-treatment or pre-concentration which cannot be done easily in-vivo, or to utilize ‘lab-on-chip’ technologies that are larger and cumbersome and often with shorter operational lifetimes such that in-vivo placement (implantation) is impractical.

Some more detailed background information is relevant with respect to the challenges of continuous extraction and sensing of ISF. Numerous attempts have been made at using ‘no pore’ or ‘no needle’ approaches. For example, techniques such as electro-osmosis (reverse iontophoresis) or vacuum suction and blister formation have been extensively studied. The problem with these approaches is that pure ISF is not extracted. Much of the ISF and analytes must travel advectively through paracellular pathways which have tight-junctions in between the cells, and as a result, many analytes in the ISF are diluted, especially larger analytes. Furthermore, these methods cause significant stress on the skin over time, and in the case of vacuum, can cause delamination of layers of the skin. Groups have also demonstrated methods whereby they create a pore through the epidermis by needles then remove the needles, or use laser or other techniques to create pores. ISF is then extracted through these pores by vacuum. However, greater skin damage is often caused with this approach. Furthermore, vacuum pressure against the skin surface will cause damage and delamination over time with long-term continuous sensing, and also requires a continuous vacuum seal to the skin which complicates the device construction and wearability (maintaining a tight seal against the body is always complex if the subject is active). With pre-formed pores, some groups have demonstrated applying a one-time positive pressure to the skin to push out ISF through pre-formed pores, but this collapses the dermis and alters the important pressure balance between blood, ISF, and lymphatic fluid, which can skew analyte concentrations. Furthermore, this causes a repeated sensation on the skin if continuous (repeated) ISF extraction and sensing is desired. Therefore, in general, methods that rely on no-pores or pre-formed pores in the skin are generally plagued with challenges for continuous sensing and extraction of ISF.

Hollow microneedles are an attractive solution for creating a sustainable pore between the dermis and an ex-vivo sensing. However, no group has ever demonstrated continuous ISF extraction using microneedle technology, clearly indicating that inventive and enabling aspects are missing. Furthermore, even before device designs and challenges can be discussed, there are fundamental limits based on physiology that should be considered if high-quality ISF extraction and sensing is to be achieved for all analytes in ISF. Assume a dermal clearance rate back through the lymphatic system of Q=8E-6 cm³/cm³−s=1/s. This rate itself is an important limit because blood is under positive pressure and ISF under negative pressure due to the lymphatic clearance, and any additional extraction of ISF by a device will alter this balance and result in additional dilution of large analytes, and/or possible collapse of the dermal compartment, and/or flow reversal from lymph back into ISF. Assume the dermis is 1.5 mm thick and 30% of that volume is collagen, for a total fluid thickness of 1 mm. This results in roughly ˜70 μL/cm² for the ISF volume in the dermis. Next, assume a collection area of 1 cm² (impractical, but allows for easy calculation). Based on lymphatic clearance rate alone, Q=0.8 nL/s for 1 cm² area, or 48 nL/min for 1 cm² area. For a more practical microneedle array of 6×6 mm² or roughly ⅓rd of a cm², the lymphatic-limited extraction rate for that array would be 17 nL/min. These would be an incredibly small extraction rates, an extraction rates that is not practically useful with current device techniques proposed or demonstrated for continuous ISF extraction.

If it is tolerable to skew the pressure balance between blood, ISF, and lymph, then hollow microneedles could extract ISF at even higher rates by moving to a regime that is limited by capillary blood flow to the dermis. Assume a blood flow rate to the dermis that is Q=22E-4/s (Kasting 2012). The revised fluid extraction rate would be 17 nL/min*22E-4/8E-6=4.7 L/min for the 6×6 mm example (0.36 cm²). This simple calculation assumes that all the blood flow to the dermis could be perfused into ISF, which is not necessarily the case in practice. Furthermore, although the capillaries are highly porous and their release of fluid content will increase as negative pressure is applied to the dermis, the dilution of large analytes will also increase since many large analytes travel paracellularly through the capillary walls. However, dilution could be more tolerable in a continuous sensing paradigm if (1) the fluid extraction rate is fairly constant, or (2), if the fluid extraction rate is not constant (vasodilation vs. vasoconstriction, exercise, skin temp, etc.) but the amount of dilution could be measured in some manner.

The 4.7 μL/min rate of extraction for the 6×6 mm example (0.36 cm²), as calculated above, are comparable to those seen experimentally for the case of holes perforated into the skin and applying vacuum. However, as noted previously, vacuum directly onto the skin surface creates additional challenges. As a result, the above calculation is still imperfect, because the specific design of the extraction device such as hollow microneedles will have a strong, if not dominant, impact on the rate at which ISF can be extracted. It should be noted, that in some cases pulsatile (periodic) extraction might be proposed, but if one is flow-rate-limited in terms of the rate of replenishment of ISF in the dermis, pulsatile extraction does not resolve this physiological limitation and does not improve the amount of sample that can be extracted over time (i.e. you are still limited to an ‘average’ extraction rate regardless if you extract periodically or continuously). In fact, pulsatile can make things worse, because during extraction, greater extraction pressures are required, which will have greater impact on potential dilution of large analytes and on possible reversal of flow of lymph back into ISF.

The specific impacts and challenges with device design are discussed next. Again consider a hollow microneedle array of 100 active needles into ˜0.36 cm² area. Assume the hydraulic conductivity of the dermis is K′=5E-16 m⁴/N−s and assume the fluid resistance imparted by the dermis dominates over fluid resistance in the device which extracts the ISF (a very reasonable assumption in many cases). Adopting a point contact-resistance formula for electronics (because fluidic resistance and electronic resistance exhibit similar behaviors), the resistance at the tip of a hollow microneedle with significant wall thickness can be calculated as K=K′*4a, where a is the radius of the opening of the microneedle. Assume for the hollow microneedles that a=40 μm. The resistance at the tip of a single microneedle inside of human dermis is therefore K=8E-20 m⁵/N-s. Next, consider next two pressures, one vacuum, one osmotic, and calculate the resulting flow rate according to Q=P*K, where Q is flow rate and P is the applied negative pressure for extracting the ISF. For vacuum at 15 psi Q=8 pL/s at each needle, which for 60 seconds/minute and 100 needles is 48 nL/min. The same calculation for 2000 PSI osmotic pressure results in 6.7 μL/min. Interestingly, these calculations show that hollow microneedles can achieve fluid extraction rates compatible with the lymphatic-limited extraction rates and blood-capillary-flow-limited extraction rates described in the previous paragraphs. A logical next question, is why are such schemes not utilized for continuous ISF extraction and sensing? Firstly, most of ISF sensing focuses on diffusion-only based sensing (none or limited advective flow), and so design for extracted ISF is simply not considered. Secondly, most extracted ISF results rely on creating open pores in the skin, which as noted above, brings practical challenges. Thirdly, in many instances device designers did not take adequate time to consider the impacts of physiology on (1) the maximum rates that ISF can be extracted without skewing analyte concentrations in ISF, and (2) the significant impact of the very low hydraulic permeability of the dermis. Fourthly, even if a person having ordinary skill in the art were to take into consideration the points made above, applying these pumping mechanisms is not trivial. For vacuum, if any needle is exposed to a route to air, which can occur easily with any skin defects, the vacuum is broken and the pumping mechanism may break or become significantly diminished. For osmotic pressure, there may still be the same air-tight issue as exists when using vacuum pressure, and the osmotic pressure would have a very limited duration as salt concentrations would build up quickly over time in the ISF sample in the device, and therefore, the pumping rate would rapidly diminish, and/or there would be a risk of diffusing the draw solution back into the body. Furthermore, vacuum induced ‘boiling’ of the ISF can also be problematic, and may require devices to be filled with wicking or hydrogel materials to keep them wet instead of partially or fully filled with water vapor (gas).

To resolve lag times, one might consider coating the ends of microneedles with a biosensor, however, this can bring additional challenges beyond issues with lag times. For example, consider a conventional microneedle length of 300 μm which is a length that has been used to minimize perceived pain by companies such as Arkal Medical, which utilized an array of 200 hollow microneedles as reported in Journal of Diabetes Science and Technology, 2014, Vol. 8 (3) 483-487, DOI: 10.1177/1932296814526191. Increasing the number of microneedles or length of microneedles causes significant increase of perceived pain as reported in Clin J Pain 2008; 24:585-594, DOI: 10.1097/AJP.0b013e31816778f9. Next, consider that the epidermis is ˜100 μm thick on locations such as the forearm. Then consider the effects of skin defects on skin roughness (10's to 100's of μm) and of hair (˜20-200 μm thick). Lastly, consider that skin roughness (peak to valley) heights are ˜ 100 μm in young adults, and ˜200 μm or more in older adults (Skin Pharmacol Physiol 2016; 29:291-299, DOI: 10.1159/000450760). It is easily feasible that at least one microneedle will not reach the dermis and therefore not be in fluidic communication with interstitial fluid. Furthermore, motion of the body or organs or changing pressures against a device can make the problem of microneedles not being in fluidic communication with interstitial fluid even worse. Returning to the consideration of microneedles that are coated with biosensors, any microneedle not implanted properly into the dermis could give a zero or false signal. Therefore, a significant challenge exists where the biosensor must be somehow kept in constant fluidic communication with the interstitial fluid in the dermis. Furthermore, simply increasing needle length may not be relevant for many applications (pain, or chance it could insert into subcutaneous fat).

As a result, practically meaningful demonstrations of continuously extracted and sensed ISF with microneedles or needles do not exist in the literature (the literature is dominated by single one-time extraction experiments). Clearly, for ISF, additional inventive steps are required to bridge the divide between physiology and ex-vivo sample transport, sample treatment, and sensing. Inventive steps are needed, spanning: (1) very small ISF sample volumes and their effects on lag time, (2) implementing pumping strategies that reliably and continually provide adequate ISF sample extractions, and (3) in dealing with skewed analyte concentrations resulting from altering the pressure balance between blood, ISF, and lymph in the dermis.

### SUMMARY OF THE INVENTION

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

Embodiments of the disclosed invention are directed to continuous extraction and sensing of analytes in interstitial fluid. Embodiments of the disclosed invention provide sensing systems that resolve lag-time challenges when the analyte is coupled to the sensor by primarily advective flow. More specifically the present invention addresses: (1) very small ISF sample volumes and their effects on lag time; (2) implementing pumping strategies that reliably and continually provide adequate ISF sample extractions; (3) dealing with skewed analyte concentrations resulting from altering the pressure balance between blood, ISF, and lymph in the dermis.

A continuous sensing device for at least one analyte in a sample of interstitial fluid is provided. The device includes at least one ex-vivo sensor specific to the a least one analyte in interstitial fluid. The device further includes at least one sample collection component in the dermis that defines at least in part an advective pathway to transport interstitial fluid to the at least one sensor. The advective pathway is air-tight, and the device includes at least one integrated pump that applies negative pressure to cause advective transport of interstitial fluid from the dermis, to the sensor, and onto the pump.

A method of sensing an analyte in an interstitial fluid is provided. The method includes advectively transporting the interstitial fluid including the analyte from a dermis of a skin into an ex-vivo device via an air-tight advective pathway defined, at least in part, by a sample collection component. The advectively transporting the interstitial fluid is done via the air-tight advective pathway, and is promoted by a negative pressure supplied by a pump. The method further includes contacting the interstitial fluid with an ex-vivo sensor configured to specifically and continuously sense the analyte.

### BRIEF DESCRIPTION OF THE DRAWINGS

The objects and advantages of the disclosed invention will be further appreciated in light of the following detailed descriptions and drawings in which:

FIG. 1 is a cross-sectional view of a device according to an embodiment of the disclosed invention.

FIG. 2 is a cross-sectional view of a device according to an embodiment of the disclosed invention, with an alternate pumping scheme based on osmotic pressure.

FIG. 3 is a cross-sectional view of a device according to an embodiment of the disclosed invention, with an alternate feature to extract ISF such as a needle or tube.

FIG. 4 is a cross-sectional view of a device according to an embodiment of the disclosed invention, with an additional feature to measure or regulate ISF extraction rate.

FIG. 5A is a cross-sectional view of a device according to an embodiment of the disclosed invention, with an additional feature to increase ISF extraction rate.

FIG. 5B is a cross-sectional view of a device according to an embodiment of the disclosed invention, with an additional feature to increase ISF extraction rate.

### DEFINITIONS

As used herein, “interstitial fluid” means the interstitial fluid found in the dermis of the skin, which can be accessed through forming a pore into the body and by placing a foreign object into the body (such as a needle or microneedle or tube other material). Because of the presence of local capillaries, in some instances when accessing interstitial fluid with techniques such as microneedles, some or blood or lymph or other biofluid may also be accessed. However, because interstitial fluid is the target fluid, even if some other fluid is mixed in, the overall fluid will still be referred to herein as interstitial fluid.

As used herein, “ex-vivo” means outside the body or not placed directly within the body. For example, a sensor placed above the epidermis of the skin is ex-vivo.

As used herein, “sample” means an collected volume of interstitial fluid as a source of analytes.

As used herein, “sample volume” means the effective total volume, or portions of volumes that form a total volume, between an ex-vivo sensor and interstitial fluid which effects the advectively-determined lag-time between concentration of an analyte in the biofluid and the concentration at the sensor. This sample volume could be a fluidic or microfluidic volume defined by walls such as channel walls or be defined by a defined fluidic pathways such as that through wicking materials such as a hydrogel.

As used herein, “sampling rate” means the effective rate at which ISF, on average, is brought into a device and transported to a sensor. For example, a sampling rate could be 20 nL/min or 1 μL/min.

As used herein, “continuous sensing” or “continuous monitoring” means the capability of a device to provide at least one measurement of an analyte in an invasive biofluid determined by a continuous or multiple collection and sensing of that measurement or to provide a plurality of measurements of the analyte over time.

As used herein, “affinity-based sensor” means as biosensor that is a continuous sensor with a plurality of probes that reversibly bind to an analyte, which do not consume, metabolize, or otherwise chemically alter the analyte, wherein the binding of analyte to the sensor increases with increasing concentration of the analyte, and the binding of the analyte decreases with decreasing concentration which then changes the sensor signal. As utilized herein, the term affinity-based sensor also means there is no need for regeneration of the sensor. For example, an aptamer-based sensor is an affinity-based sensor because it can release analyte without regeneration, whereas an antibody-based sensor is not.

As used herein, “microfluidic components” are channels in polymer, textiles, paper, hydrogels, or other components known in the art of microfluidics for guiding movement of a fluid or at least partial containment of a fluid.

As used herein, “diffusion” is the net movement of a substance from a region of high concentration to a region of low concentration. This is also referred to as the movement of a substance down a concentration gradient.

As used herein, “advective transport” is a transport mechanism of a substance or conserved property by a fluid due to the fluid's bulk motion.

As used herein, “convection” is the concerted, collective movement of groups or aggregates of molecules within fluids and rheids, either through advection or through diffusion or a combination of both.

As used herein, “lag time” is the time it requires for a change in analyte concentration in interstitial fluid, to reach a sensor by mainly advective transport through a microfluidic pathway, such that the volume of fluid immediately adjacent to the sensor is at 90% of the concentration of the concentration in the invasive biofluid. The term ‘mainly’ means the majority of the analyte. For example, if the analyte increases by 2× concentration in the biofluid over a period of 10 minutes, the majority of the increased analyte concentration at the sensor will be caused by advective flow.

### DETAILED DESCRIPTION OF THE INVENTION

Embodiments of the disclosed invention are directed to continuous ex-vivo affinity-based sensing of analytes in interstitial fluid. Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be sensors such as electrochemical aptamer sensors that sense analytes such as cortisol, vasopressin, or IL-6, for example. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention.

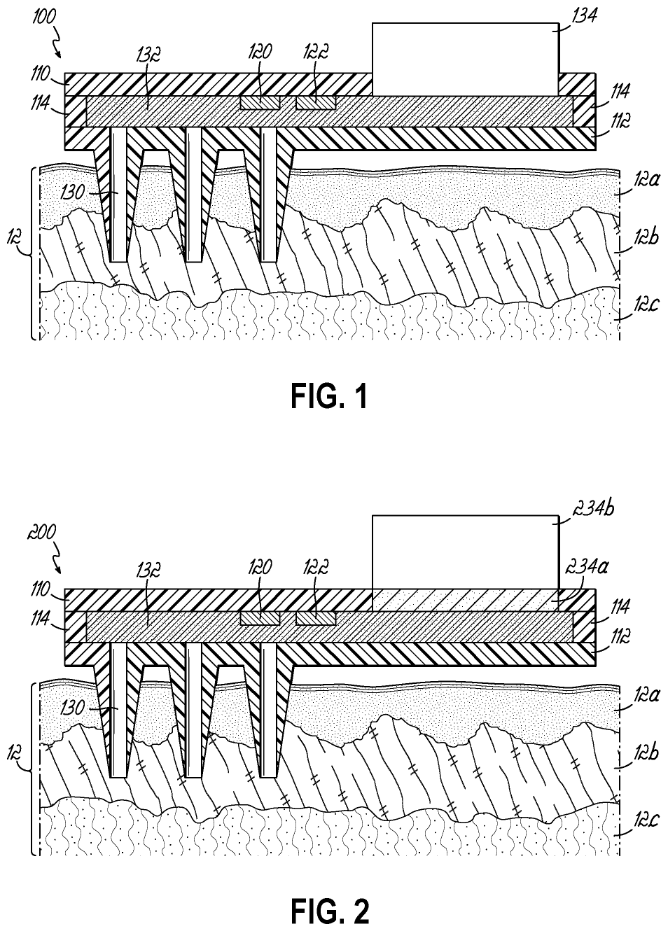

With reference to FIG. 1, in an embodiment of the disclosed invention, an ex-vivo device 100 is placed partially in-vivo into the skin 12 comprised of the epidermis 12a, dermis 12b, and the subcutaneous or hypodermis 12c. A portion of the device 100 accesses interstitial fluid from the dermis 12b. Access is provided, for example, by microneedles 112 defined by a material formed of metal, polymer, semiconductor, glass, or other suitable material, and may include at least one pore or a hollow lumen 130 that contributes to a sample volume. Sample volume is also contributed to by volume 132 between the microneedles 112 and a sensor 120 and/or sensor 122. The device also includes a pump 134 that applies a pressure to extract ISF, and the volume between the pump 134 and the sensors 120 or 122 does not contribute to the total sample volume because after sensor 120 or 122, the analytes have already been detected by sensor 120 or 122 and then are simply transported onto the pump 134 by advective flow through a microfluidic pathway. The device includes materials 110, 114 which properly seal the device, such as polymers, metals, or glass. This seal should be suitably air-tight, or compensated for by other means that will be described later, so that pump 134 may properly extract ISF without entry of air that could cause device malfunction (as will be discussed later).

With further reference to FIG. 1, an example can be taught as follows. Consider a hollow microneedle 112 array of 100 active microneedles covering ˜0.36 cm² area. Assume the hydraulic conductivity of the dermis is K′=5E-16 m⁴/N-s and assume the fluid resistance imparted by the dermis 12b dominates over fluid resistance in the device 100 which extracts the ISF. Assume the microneedles 112 have a hollow lumen 130 with a radius of 40 μm, and the resistance at the tip of the microneedle inside of human dermis is K=8E-20 m⁵/N-s. Assume the pump 134 is a vacuum pump or vacuum reservoir at 9.4 psi pressure relative to the dermis 12b resulting in Q=5 pL/s at each microneedle 112, which for 100 needles is 30 nL/min total sampling rate. This is a very small flow rate. Assume volume 132 is a channel of dimension 0.36 cm² and 10 μm thickness contributing 360 nL to a total sample volume, and this volume 132 contains sensor 120 that is a thin gold electrode functionalized with aptamer probes for IL-6, as an example. Assume the 100 microneedles are 350 μm long and 40 μm in radius, contributing 100*350E-4*3.14*(40E-4) 2 cm³ or a volume of 176 nL to a total sample volume. The total sample volume for the hollow lumen 130 plus volume 132 is therefore ˜536 nL and at 30 nL/min sampling rate would create an approximate lag time of ˜18 minutes, or less than 20 minutes. Achieving a small volume 132 could be challenging with a 10 μm height, so scaling the channel dimensions (height) to 20 μm, 50 μm, or 100 μm would result in approximate sample volumes that are <600 nL, <1200 nL, 3000 nL, <6000 nL, and lag times that are less than 40, 100, or 200 minutes. Next, assume multiple pores are provided in the microneedles 112, and/or more microneedles 112 are used, and/or the microneedles 112 are constructed of a semi-porous material such as a porous ceramic, such that cumulatively the microneedle 112 collection area in the dermis 12b is increased by 5×, 10×, or 100× compared to microneedles 112 without pores and/or fewer microneedles 112, and the sampling rate is increased to 150 nL/min, or 300 nL/min, or 3 μL/min. As a result, the lag times are less than 5, 10, 20, 50, 100 minutes or 1, 2, 4, 10, 20 minutes or 0.1 0.2, 0.4, 1, or 2 minutes. Generally, with microneedle sampling devices, fewer microneedles are preferred because devices with fewer microneedles cause less potential irritation of the skin. Therefore, the amount of microneedles 112 could also be <200, <100, <50, <20, <10 or even <5 microneedles and with the higher porosity microneedle 112 designs taught above still provide adequate lag times. The area of the device 100 could also scale with the number of microneedles 112, such that the sample volume is, for example, <300 nL, <120 nL, or <60 nL. As noted in the background section, if too much ISF is extracted, analyte concentrations in the sample ISF can become skewed, and therefore at the above mentioned 30, 150, 300, or 3000 nL/min and 0.36 cm² device area, the device can operate using sampling rates that are <100 nL/min/cm², <500 nL/min/cm², <1 μL/min/cm², or <10 μL/min/cm².

With further reference to FIG. 1, the above calculation assumes that every microneedle 112 is sealed against the skin with a perfect vacuum seal. Any air that could reach a microneedle 112 pore could result in breaking of vacuum and malfunctioning of the device 100. Therefore, volume 132 above the microneedles 112 could be filled with at least one air-blocking material or component (not shown in FIG. 1) such as a wicking material such as silica powder, fumed-silica, cellulose, hydrogels such as agar, gelatin, or other suitable materials that have a wicking pressure that is greater than the applied pressure from the pump 134. To ensure that fluid resistance inside the device is not increased too much, the air-blocking material could also be a thin film (e.g. 1-10 μm of agar or gelatin or fumed-silica coated onto a track-etch membrane that provides mechanical strength). For such materials to function properly with a wicking or osmotic driven flow, they must be wet with ISF or at least initially wet with another fluid such as propylene glycol or glycerin. This raises an issue with shelf-storage of the device 100. If a vacuum reservoir 134 is to be utilized, the reservoir should not receive air from outside until the device 100 is ready for use. For example the device 100 could be shelf-stored dry in a vacuum pouch, void of fluid, and the microneedle tips coated with a dissolvable air-blocking material such as sucrose or polyvinyl alcohol. Therefore, unless a microneedle 112 becomes wet, the microneedle 112 could not provide a pathway for air to reach vacuum reservoir or pump 134. This could also resolve the need for air-blocking during operation of the device 100 (e.g. a microneedle 112 that was not into the dermis 12b would not be wet and therefore not be opened to allow air to enter the microneedle 112). In yet another embodiment of the present invention, hollow lumen 130 and volume 132 can be channels or pores, or be filled with an air blocking material such as a wicking fiber, gel, or other material to suppress water vapor or air-bubble formation within the device 100 itself due to the negative pressure applied by the pump 134.

With further reference to FIG. 1, pump 134 could also be a wicking material such as sodium acrylate, silica gel, cellulose, or other suitable material that provide at least >1 psi, >2 psi, >5 psi, >10 psi wicking pressure.

With further reference to FIG. 1, pump 134 could have a volume or wicking capacity of, for example, but not limited to >10 μL, >100 μL, >500 μL, >1 mL, or >5 mL such that at 30 nL/min sampling rate, the device 100 could continuously extract ISF for at least one of >300 minutes, >3000 minutes, >15000 minutes, >30,000 minutes, >150,000 minutes. Accordingly, at 300 nL/min, sampling rate the continuously extract ISF for at least one of >60 minutes, >600 minutes, >3000 minutes, >6000 minutes, >30,000 minutes. Accordingly, at 300 nL/min, sampling rate the device could continuously extract ISF for at least one of >30 minutes, >300 minutes, >1500 minutes, >3000 minutes, >15,000 minutes. For prolonged continuous vacuum operation for an hour or more, the partial vapor pressure of water in the pump 134 (evaporation and water vapor pressure in pump 134) could be problematic and reduce the applied vacuum pressure. Therefore, pump 134 may also include at least one desiccant such as silica powder other suitable material. Even with a desiccant, water vapor may be an issue over time, and pump 134 may also be a mechanical pump, or other suitable pump, to continually provide pressure. An alternate osmotic pump will be taught next that may be the most preferred given its ability to apply stronger pressures and for prolonged periods of time.

With reference to FIG. 2, were like numerals refer to like features in other embodiments of the present invention, a device 200 includes an alternate pumping scheme 234 based on osmotic pressure. With a conventional osmotic pump, membrane 234a, for example, would be impermeable to small ions and solutes, and as a result the concentration of such small ions and solutes would build up over time in volume 232 and eventually cause device malfunction or interfere with sensing of an analyte by sensors 220 or 222. This limitation is unfortunate, because osmotic pressures can be much greater than vacuum pressures (10X-1000X) and therefore enable higher sampling rates into the device 200. The device 200 utilizes an ion-porous membrane 234a which functions as follows. A portion of the pump 234 is configured to draw fluid into the device 200 by osmosis into a feed or sample solution 234b, containing at least one solvent such as water, and to draw in by advective flow ionic species such as Cl−, Na+, OH−, H−, K+, NH₄+, lactate, urea, etc. An osmotic pressure is generated by the draw solution 234b that contains at least one polyelectrolyte or other draw solute that increases the osmolality of draw solution 234b compared to ISF in the device 200. Membrane 234a could be a nanofiltration membrane, dialysis, membrane, or other suitable membrane 234a that is an ion-porous membrane. The membrane is porous enough that ionic solutes in interstitial fluid, including even proteins with some membranes, are drawn into 234b by advective flow, but the draw solute is large enough such that it is unable to traverse the membrane 234a and therefore is trapped in the solution of 234b. Draw solutes that satisfy this requirement include but are not limited to polyelectrolytes. Linear polyelectrolytes are solids at room temperature while branched polyeletrolytes are liquids. Linear polyethyleneimines are soluble in hot water, at low pH, in methanol, ethanol, or chloroform. Linear polyethyleneimines are insoluble in cold water, benzene, ethyl ether, and acetone. They have a melting point of 73° C.-75° C. They can be stored at room temperature. In an example embodiment of the present invention, the draw solution 234b are shelf-stable and ready to use. For example, 234b could be a liquid and require little or no-solvent, which is the case for branched polyelectrolytes. For example, 234b could be a linear polyelectrolyte and dissolved in a solution of ethanol. For example, polyelectrolytes can include, but are not limited to: polyacrylic acids, polysulfonic acids, polyimidazoles, polyethylenimines, chitosan (cationic) and sodium alginate (anionic). The draw solute can also be immobilized by being cross-linked to itself or grafted or cross-linked or bonded to a scaffold such as a hydrogel or such as an aerogel that would allow for dry storage of element 234b. If the draw solute is immobilized, membrane 234a can also be optional. A result of this embodiment of the present invention is that an osmotic pump can be utilized that can continuously pump a sample of ISF without significantly increasing ISF sample osmolality near sensors 120, 122. For example, osmolality of the ISF sample near sensors 120, 122, could be increased by <10%, <50%, <100%, <500%, or <1000% depending on the choice of membrane utilized (tighter, less porous membranes, will cause larger increases in osmolality, as for example by using membranes with lower molecular weight cutoffs).

With further reference to FIG. 2, with a proper osmotic pumping mechanism now taught, at different molar concentrations of draw solutes in 234b the draw pressure could be >2000 psi, or with reduced draw solute concentrations in a range of >5 psi, >10 psi, >20 psi, >50 psi, >100 psi, >200 psi, >500 psi, >1000 psi, >2000 psi. Performing a calculation similar to that of FIG. 1, with the lowest needle porosity taught for FIG. 1 (a single hollow needle), but with these greater pressures, sampling rates of up to >6.7 μL/min can be achieved, or >15 nL/min, >30 nL/min, >60 nL/min, >150 nL/min, >300 nL/min, >600 nL/min, >1500 nL/min, >3000 nL/min, or >6000 nL/min. These sampling rates all scale accordingly if the porous collection area of the needles are increased, and/or more needles are increased, scaling the sample rates by 5×, 10× or even 100×, such that even, for example, >30 μL/min, >60 μL/min, or >6 mL/min are possible. However, as taught in the background section, physiology of the dermis places a limit such that maximum extraction rates of available ISF are 10's of nL to 10's of μL/cm² at most based on ISF sample quality and ISF sample availability in the dermis, and therefore the device 200 area must be scaled accordingly if larger sampling rates are desired. The volume of the pump 234 can be scaled also as taught for the pump 134 of FIG. 1. For example, a 1 mL osmotic pump 234 could operate at 90% of pumping capacity until 100 μL of ISF fluid is brought into the pump 234, which for a pumping rate of 100 nL/min would allow 100 minutes of operation.

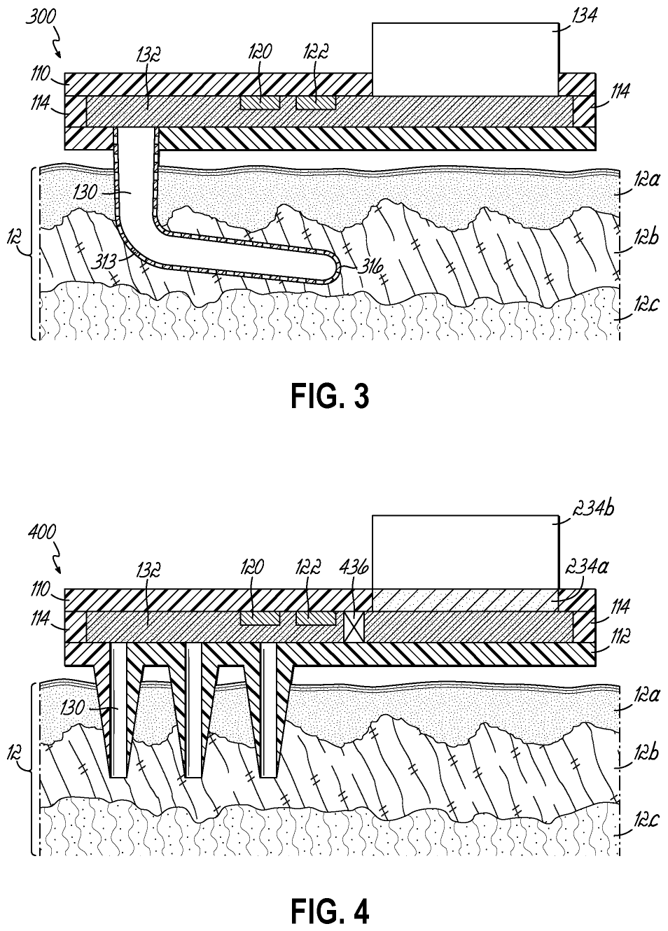

With reference to FIG. 3, were like numerals refer to like features in other embodiments of the present invention, a device 300 includes a needle or tube with a material border 316. The material border 316 could be a metal such as stainless steel with numerous holes or apertures machined into the needle by laser, chemical, mechanical, or other means to allow ISF to enter into the device 300 by advective flow. The material border 316 could also be a dialysis or nanofiltration or membrane tubing that allows ISF and analytes to enter into the device 300 by advective flow. FIG. 3 simply teaches that the present invention is not limited to just microneedles and should be more broadly interpreted as including at least one sample collection component 313 in the dermis 12b, that sample collection component 313 being microneedles, needles, tubes, or other suitable means that satisfy the other elements of operation for the present invention.

With reference to FIG. 4, where like numerals refer to like features in other embodiments of the present invention, a device 400 includes at least one sampling rate component 436, which regulates or measures sampling rate. For measuring sampling rate, the component 436 could be a sampling rate measurement component such as a thermal mass flow sensor. For regulating sampling rate, the component 436 could be a sampling rate regulating component, such as a microfluidic valve, or a simple flow restriction or a slug of hydrogel that is >30 wt. %. For example, if the hydraulic resistance of the component 436 was 10× greater than the hydraulic resistance of the microneedle openings in the dermis, as previously taught, the sampling rate through the device would be regulated and constant due to the sample rate component 436, regardless of conditions in the dermis, and/or how many microneedles might be malfunctioning (i.e. the fluidic pressure drop across the microfluidic pathway in the device 400 would dominantly be across component 426 during operation of the device). This is important, because, as taught in the background section, low ISF extraction rates may be preferred in order to not skew analyte concentrations and/or the ISF extraction rates should be fairly constant such that if analyte dilution occurs the dilution will be fairly constant in ISF and not perturbed by changes in ISF extraction rate. Said differently, the sampling rate could be regulated such that it does not change by more than 5, 10, 20, 50, or 100% during operation of the device 400. With use of a flow restriction element for the sampling rate component 436, a 2× or 10× reduction in sampling rate, the previous mentioned sampling rates of <100 nL/min/cm², <500 nL/min/cm², <1 μL/min/cm², or <10 μL/min/cm², could be extended to a range of <10 nL/min/cm², <50 nL/min/cm², <100 nL/min/cm², <500 nL/cm², <1 L/min/cm², <5 L/min/cm², or <10 μL/min/cm².

With further reference to FIGS. 1-4 and embodiments of the present invention, in some cases the dilution of analytes will be less predictable or controllable based on physiology (e.g. vasoconstriction or vasodialation of capillaries), or will be less predictable because the sampling rate in the device will be variable due to variable pressure by a pump 134, variable access to ISF such as microneedles coming loose from skin, or other factors. In such cases, two or more sensors can be utilized to measure dilution and therefore provide accurate concentration readings. For example, sensor 120 could measure Human Chorionic Gonadotropin which has a molecular weight of 36 kDa, and sensor 122 could measure albumin which has a molecular weight of 66.5 kDa. Albumin has a steady concentration in blood, and therefore its measure in ISF gives a measure of the amount of dilution in ISF for albumin and therefore other large analytes as well such as Human Chorionic Gonadotropin. Therefore, the present invention may utilize two or more sensors to measure two or more analytes to compare their ratios. Therefore, the present invention may utilize at least one sensor that measures analyte dilution in ISF by measurement of at least one analyte that is diluted in ISF. Even larger analytes such as antibodies can be measured, which have stronger dilution effects. This ratio measurement is also important for measuring antagonistic analytes (where in a physiological condition the analytes change concentration in opposite ways).

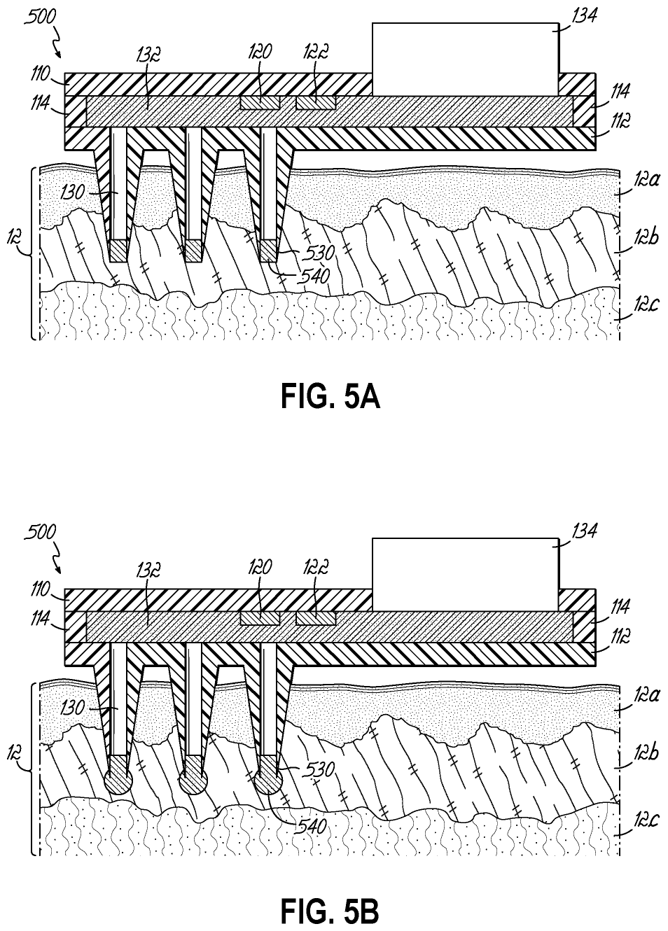

With reference to FIGS. 5A and 5B, where like numerals refer to like features in other embodiments of the present invention, a device 500 further includes at least one swellable component 540. Swellable component 540 may be a swelling hydrogel. Swellable component 540 addresses a challenge in the interfacial area between the dermis and needle, without dramatically increasing microneedle 112 size. The device 500 may have the tip 530 of the needle impregnated with the swellable component 540, such as a swelling hydrogel such as polyacrylamide or other suitable hydrogel. Once inserted into the dermis 12b the swelling could increase the effective area of contact with the dermis 12b, therefore lowering the hydraulic resistance. Additionally, the swellable component 540 may shift a collagen matrix, preventing the formation of clogging at the needle inlet, which may be located at the tip 530. The tip 530 could also be coated externally with the swellable component 540, which may be a hydrogel, to achieve a similar effect.

With further reference to FIGS. 1-4 and embodiments of the present invention, if a device was missing elements such as 114 or was not perfectly sealed, then vacuum or osmotic pressure could draw in air into the device causing malfunction. This can be resolved, however, if the microfluidic pathway between the dermis, sensors, and pump are filled appropriately with wicking materials with a wicking strength that is greater than the pumping pressure applied by the pump to the wicking material. As a result, the present invention may include an open or partially open microfluidic design. Wicking materials may include hydrogels, fumed silica, or other suitable wicking materials that once wet, stay wet, even if a strong negative pressure is applied to them such as vacuum or osmotic pressure.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

1. A continuous sensing device for at least one analyte in a sample of interstitial fluid, comprising;
at least one ex-vivo sensor specific to the a least one analyte in interstitial fluid;
at least one sample collection component in the dermis that defines at least in part an advective pathway to transport interstitial fluid to the at least one sensor;
wherein the advective pathway is air-tight, and;
at least one integrated pump that applies negative pressure to cause advective transport of interstitial fluid from the dermis, to the sensor, and onto the pump.

2. The device of claim 1, further comprising at least one affinity-based biosensor.

3. The device of claim 2, wherein said affinity-based biosensor is an electrochemical aptamer-based sensor.

4. The device of claim 1, wherein the affinity-based biosensor is an optical aptamer sensor.

5. The device of claim 1, wherein the advective pathway between the at least one sensor and the dermis has a sample volume that is at least one of <60 nL, <120 nL, <300 nL, <600 nL, <1200 nL, <3000 nL, <6000 nL.

6. The device of claim 1, further comprising a lag time that is at least one of <1 minute, <5 minutes, <20 minutes, <100 minutes.

7. The device of claim 1, wherein the advective pathway includes at least one microneedle.

8. The device of claim 1, wherein the number of at least one microneedles is <5, <10, <20, <50, <100, <200.

9. The device of claim 1, wherein the advective transport of interstitial fluid from the dermis to the at least one sensor is characterized by a sampling rate that is <50 nL/min/cm², <100 nL/min/cm², <500 nL/min/cm², <1 μL/min/cm², or <10 μL/min/cm².

10. The device of claim 1 further comprising at least one air-blocking component.

11. The device of claim 10 wherein the at least one air-blocking component has a first wicking pressure, and wherein the pump has a second pressure it applies to the air-blocking component, and wherein the first wicking pressure is greater than the second pressure.

12. The device of claim 10 wherein the at least one air-blocking component is initially wet with a fluid other than interstitial fluid.

13. The device of claim 10 wherein the air-blocking component is dissolvable.

14. The device of claim 1 wherein the pump is a vacuum.

15. The device of claim 14, wherein the pump further includes at least one desiccant.

16. The device of claim 1 wherein the pump is a wicking material.

17. The device of claim 1 wherein the pump has a pressure of at least one of >1 psi, >2 psi, >5 psi, >10 psi.

18. The device of claim 1 wherein the pump is an osmotic pump.

19. The device of claim 18 wherein the pump further includes at least one ion-porous membrane and at least one draw-solute that is unable to traverse said ion-porous membrane.

20. The device of claim 18 wherein the pump further includes at least one draw-solute that is immobilized.

21. The device of claim 18 wherein the sample of interstitial fluid adjacent to the at least one sensor has an osmolality that is at least one of <10%, <50%, <100%, <500%, or <1000% greater than the osmolality of interstitial fluid in the dermis.

22. The device of claim 18 wherein the osmotic pump applies a pressure of at least one of >5 psi, >10 psi, >20 psi, >50 psi, >100 psi, >200 psi, >500 psi, >1000 psi, >2000 psi.

23. The device of claim 18 wherein the osmotic pump applies a pressure that creates an advective flow that is least one of >15 nL/min, >60 nL/min, >150 nL/min, >600 nL/min, >3000 nL/min, or >6000 nL/min, 30 μL/min, >60 μL/min.

24. The device of claim 1 wherein the pump has a pumping duration of at least one of >30, >60, >300, >600, >1500, >3000, >6000, >15,000 minutes.

25. The device of claim 1 further comprising at least one sampling rate measurement component.

26. The device of claim 1 further comprising at least one sampling rate regulating component.

27. The device of claim 26 further comprising a sampling rate that during operation of the device does not change by more than 5, 10, 20, 50, or 100%.

28. The devices of claim 1 further comprising a first sensor for measuring concentration of a first analyte and a second sensor for measuring concentration of a second analyte, wherein the first sensor and second sensor provide together a ratio of concentration of the first analyte to the second analyte.

29. The device of claim 28 wherein the ratio further provides a measure of dilution of at least one analyte in interstitial fluid compared to the concentration of the analyte in blood.

30. The device of claim 1 wherein the advective pathway that is air-tight is at least in part air-tight due to at least one wicking material with a wicking strength that is greater than the pressure applied by the pump to the wicking material.

31. The device of claim 1 further comprising at least one swellable component that decreases hydraulic resistance at least where the device interfaces with the dermis.

32. The device of claim 1, wherein the at least one sample collection component has a length of 300 micrometers.

33. A method of sensing an analyte in an interstitial fluid, the method comprising:
advectively transporting the interstitial fluid from a dermis of a skin into an ex-vivo device via an air-tight advective pathway defined, at least in part, by a sample collection component, the interstitial fluid including the analyte, wherein advectively transporting the interstitial fluid via the air-tight advective pathway is promoted by a negative pressure supplied by a pump;
contacting the interstitial fluid with an ex-vivo sensor configured to specifically and continuously sense the analyte.

34. The method of claim 33, wherein the ex-vivo sensor includes an affinity-based biosensor.

35. The method of claim 34, wherein said affinity-based biosensor is an electrochemical aptamer-based sensor.

36. The method of claim 33, wherein the affinity-based biosensor is an optical aptamer sensor.

37. The method of claim 33, wherein the air-tight advective pathway between the at least one sensor and the dermis has a sample volume that is at least one of <60 nL, <120 nL, <300 nL, <600 nL, <1200 nL, <3000 nL, <6000 nL.

38. The method of claim 33, wherein a time to perform advectively transporting the interstitial fluid from the dermis to the ex-vivo sensor includes a lag time that is at least one of <1 minute, <5 minutes, <20 minutes, <100 minutes.

39. The method of claim 33, wherein the advective pathway includes at least one microneedle.

40. The method of claim 33, wherein the number of at least one microneedles is <5, <10, <20, <50, <100, <200.

41. The method of claim 33, wherein the advective transport of interstitial fluid from the dermis to the at least one sensor is characterized by a sampling rate that is <50 nL/min/cm², <100 nL/min/cm², <500 nL/min/cm², <1 μL/min/cm², or <10 μL/min/cm².

42. The method of claim 33 further comprising at least one air-blocking component.

43. The method of claim 42 wherein the at least one air-blocking component has a first wicking pressure, and wherein the pump has a second pressure the pump applies to the air-blocking component, and wherein the first wicking pressure is greater than the second pressure.

44. The method of claim 33 wherein prior to advectively transporting the interstitial fluid, the at least one air-blocking component is wet with a fluid other than the interstitial fluid.

45. The method of claim 42 wherein the air-blocking component is dissolvable.

46. The method of claim 33 wherein the pump is a vacuum.

47. The method of claim 46, wherein the pump further includes at least one desiccant, and wherein the method further comprises adsorbing water via the desiccant.

48. The method of claim 33 wherein the pump is a wicking material.

49. The method of claim 33 wherein the pump has a pressure of at least one of >1 psi, >2 psi, >5 psi, >10 psi.

50. The method of claim 33 wherein the pump is an osmotic pump.

51. The method of claim 50 wherein the pump further includes at least one ion-porous membrane and at least one draw-solute that is unable to traverse said ion-porous membrane.

52. The method of claim 50 wherein the pump further includes at least one draw-solute that is immobilized.

53. The method of claim 50 wherein the sample of interstitial fluid adjacent to the at least one sensor has an osmolality that is at least one of <10%, <50%, <100%, <500%, or <1000% greater than the osmolality of interstitial fluid in the dermis.

54. The method of claim 50 wherein the osmotic pump applies a pressure of at least one of >5 psi, >10 psi, >20 psi, >50 psi, >100 psi, >200 psi, >500 psi, >1000 psi, >2000 psi.

55. The method of claim 50 wherein the osmotic pump applies a pressure that creates an advective flow that is least one of >15 nL/min, >60 nL/min, >150 nL/min, >600 nL/min, >3000 nL/min, or >6000 nL/min, 30 μL/min, >60 μL/min.

56. The method of claim 33 wherein the pump has a pumping duration of at least one of >30, >60, >300, >600, >1500, >3000, >6000, >15,000 minutes.

57. The method of claim 33 further comprising at least one sampling rate measurement component.

58. The method of claim 33 further comprising at least one sampling rate regulating component.

59. The method of claim 58 further comprising a sampling rate that during operation of the method does not change by more than 5, 10, 20, 50, or 100%.

60. The method of claim 33 further comprising a first sensor for measuring concentration of a first analyte and a second sensor for measuring concentration of a second analyte, wherein the first sensor and second sensor provide together a ratio of concentration of the first analyte to the second analyte.

61. The method of claim 60 wherein the ratio further provides a measure of dilution of at least one analyte in interstitial fluid compared to the concentration of the analyte in blood.

62. The method of claim 33 wherein the air-tight advective pathway is at least in part air-tight due to at least one wicking material with a wicking strength that is greater than the negative pressure applied by the pump to the wicking material.

63. The method of claim 33, wherein the at least one sample collection component has a length of 300 micrometers.
